(19) United States
(12) Patent Application Publication
(10) Pub. No.: US 2022/0369952 A1
(43) Pub. Date: Nov. 24, 2022

(54) DISEASE DIAGNOSIS DEVICE AND DIAGNOSIS METHOD THEREOF

(71) Applicant: Electronics and Telecommunications Research Institute, Daejeon (KR)

(72) Inventors: Dae-Sik LEE, Daejeon (KR); Myungjoon KWACK, Daejeon (KR); Hyung Ju PARK, Daejeon (KR); Hyung-Kun LEE, Daejeon (KR); Doyeob KIM, Daejeon (KR); Jae Eun LEE, Daejeon (KR)

(73) Assignee: Electronics and Telecommunications Research Institute, Daejeon (KR)

(21) Appl. No.: 17/735,343

(22) Filed: May 3, 2022

(30) Foreign Application Priority Data
May 24, 2021 (KR) 10-2021-0066162

(51) Int. Cl. A61B 5/08; A61B 5/097; B01D 53/26; B01D 53/28; B01D 53/30; B01D 53/04

(52) U.S. Cl. CPC A61B 5/082; A61B 5/097; B01D 53/261; B01D 53/28; B01D 53/30; B01D 53/0438; B01D 53/0446; B01D 2253/1124; B01D 2253/1122; B01D 2253/20; B01D 2257/80; B01D 2257/90

(57) ABSTRACT

Disclosed are a disease diagnosis device and a diagnosis method thereof. The disease diagnosis device includes a pump for pumping a respiratory gas, a first pre-treatment portion connected to the pump and removing moisture and bad breath in the respiratory gas, and a volatile organic compound (VOC) detection portion connected between the first pre-treatment portion and the pump to detect VOCs in the respiratory gas.

### CROSS-REFERENCE TO RELATED APPLICATIONS

This U.S. non-provisional patent application claims priority under 35 U.S.C. § 119 of Korean Patent Application No. 10-2021-0066162, filed on May 24, 2021, the entire contents of which are hereby incorporated by reference.

### BACKGROUND

The present disclosure herein relates to a disease diagnosis device and a diagnosis method thereof, and more particularly, to a disease diagnosis device for lung cancer and a diagnosis method thereof.

In general, a gas sensor is applied to a wide range of fields such as environmental monitoring, occupational safety, a breathalyzer and a food industry. In the future, the gas sensor may be applied to new fields such as a medical industry and a space industry. The gas sensor is arranged in the form of an array and developed into an electronic nose or an electronic olfactory system.

### SUMMARY

The present disclosure provides a disease diagnosis device capable of increasing reliability for detecting volatile organic compounds in a respiratory gas.

An embodiment of the inventive concept provides a disease diagnosis device including: a pump configured to pump a respiratory gas; a first pre-treatment portion connected to the pump and configured to remove moisture and bad breath in the respiratory gas; and a volatile organic compound (VOC) detection portion connected between the first pre-treatment portion and the pump to detect VOCs in the respiratory gas. Here, the first pre-treatment portion includes: a first chamber; a moisture filter disposed in the first chamber to remove the moisture; and a bad breath filter disposed between the moisture filter and the VOC detection portion to remove the bad breath.

In an embodiment, the bad breath filter may include metal powder.

In an embodiment, the metal powder may include copper oxide or platinum.

In an embodiment, the moisture filter may include cotton.

In an embodiment, the first pre-treatment portion may further include a first heater disposed in the first chamber and surrounding the moisture filter and the bad breath filter.

In an embodiment, the disease diagnosis device may further include a second pre-treatment portion disposed between the first pre-treatment portion and the VOC detection portion to adjust moisture of the respiratory gas. Here, the second pre-treatment portion may include: a water bubbler configured to vaporize DI water; and a hygrometer disposed between the water bubbler and the VOC detection portion to detect moisture of the respiratory gas.

In an embodiment, the water bubbler may include: a water bath configured to store the DI water; and a first nitrogen supply portion connected to the water bath and configured to generate vapor of the DI water by supplying a nitrogen gas into the DI water.

In an embodiment, the disease diagnosis device may further include a third pre-treatment portion disposed between the second pre-treatment portion of the VOC detection portion to adjust non-polar VOCs of the respiratory gas. Here, the third pre-treatment portion may include: a benzene bubbler configured to vaporize a benzene solution; and a non-polar VOC gas sensor disposed between the benzene bubbler and the VOC detection portion to detect the non-polar VOCs of the VOCs.

In an embodiment, the benzene bubbler may include: a benzene bath configured to store the benzene solution; and a second nitrogen supply portion connected to the benzene bath and configured to generate the benzene gas by supplying a nitrogen gas into the benzene solution.

In an embodiment, the VOC detection portion may include a polar VOC sensor array.

In an embodiment of the inventive concept, a disease diagnosis device includes: a pump configured to pump a respiratory gas; a first intake line connected to the pump; a first pre-treatment portion connected between the first intake line and the pump and configured to remove moisture and bad breath in the respiratory gas; a volatile organic compound (VOC) detection portion connected between the first pre-treatment portion and the pump to detect VOCs in the respiratory gas; a second intake line configured to connect the first pre-treatment portion to the VOC detection portion; and a first bypass line branched from the first intake line and connected to the second intake line to bypass the first pre-treatment portion.

In an embodiment, the first intake line may have a first intake valve, the first bypass line may have a first bypass valve that is opened and closed in an opposite manner to the first intake valve, and the disease diagnosis device may further include a second intake valve disposed between the pump and the VOC detection portion.

In an embodiment, the disease diagnosis device may further include: a second pre-treatment portion disposed between the first pre-treatment portion and the VOC detection portion; a third intake line disposed between the first pre-treatment portion and the second pre-treatment portion; and a second bypass line branched from the first bypass line and connected to the second intake line to bypass the second pre-treatment portion.

In an embodiment, the disease diagnosis device may further include: a third pre-treatment portion disposed between the second pre-treatment portion and the VOC detection portion; a fourth intake line disposed between the second pre-treatment portion and the third pre-treatment portion; and a third bypass line branched from the second bypass line and connected to the third intake line to bypass the third pre-treatment portion.

In an embodiment, the third intake line may have a third intake valve, the second bypass line may have a second bypass valve that is opened and closed in an opposite manner to the third intake valve, the fourth intake line may have a fourth intake valve, and the third bypass line may have a third bypass valve that is opened and closed in an opposite manner to the fourth intake valve.

In an embodiment of the inventive concept, a disease diagnosis method includes: pumping a respiratory gas; removing moisture in the respiratory gas by using a moisture filter; removing bad breath in the respiratory gas by using a bad breath filter; and determining lung cancer of a human body by detecting volatile organic compounds (VOCs) in the respiratory gas by using a sensor array.

In an embodiment, the disease diagnosis method may further include adjusting the moisture of the respiratory gas to reference moisture by vaporizing DI water.

In an embodiment, the reference moisture may be about 40% to about 60%.

In an embodiment, the disease diagnosis method may further include adjusting a concentration of non-polar VOCs in the respiratory gas to a reference concentration by vaporizing a benzene solution.

In an embodiment, the reference concentration may be about 0.1 ppm to about 0.5 ppm.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is a cross-sectional view illustrating an example of a disease diagnosis device according to an embodiment of the inventive concept;

FIG. 2 is a perspective view illustrating an example of a sensor array of FIG. 1;

FIGS. 3A and 3B are graphs showing a lung cancer signal or a healthy signal of a respiratory gas of FIG. 1;

FIG. 4 is a cross-sectional view illustrating an example of the disease diagnosis device according to an embodiment of the inventive concept;

FIG. 5 is a cross-sectional view illustrating an example of the disease diagnosis device according to an embodiment of the inventive concept; and

FIG. 6 is a flowchart showing a disease diagnosis method according to an embodiment of the inventive concept.

### DETAILED DESCRIPTION

Hereinafter, embodiments of the present invention will be described in detail with reference to the accompanying drawings. Advantages and features of the present invention, and implementation methods thereof will be clarified through following embodiments described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Further, the present disclosure is only defined by scopes of claims. Like reference numerals refer to like elements throughout.

In the specification, the technical terms are used only for explaining a specific embodiment while not limiting the present invention. In the specification, the terms of a singular form may include plural forms unless referred to the contrary. The meaning of ‘comprises’ and/or ‘comprising’ specifies a component, a step, an operation and/or an element does not exclude other components, steps, operations and/or elements. Also, it will be understood that each of a respiratory gas, a sensor, and volatile organic compounds in this specification has a meaning generally used in the biology field. Since preferred embodiments are provided below, the order of the reference numerals given in the description is not limited thereto.

The above-described features are detailed embodiments for implementing the present invention. The present invention may include embodiments that are easily changed or simply changed in design in addition to the above-described embodiments. The present invention may also include technologies that are easily changed and implemented by using the above-described embodiments.

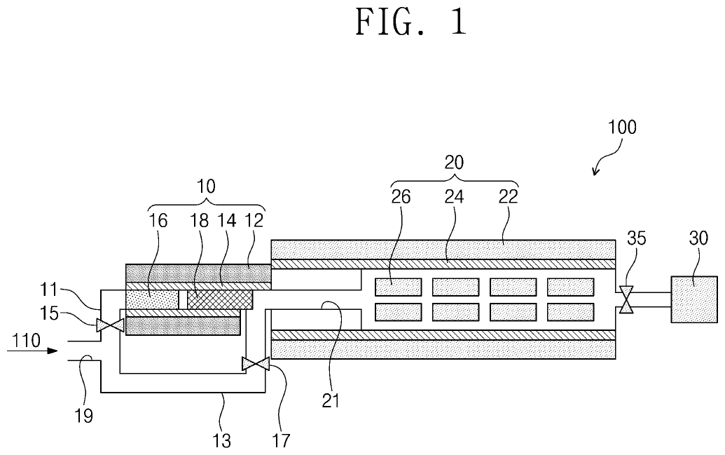

FIG. 1 is a view illustrating an example of a disease diagnosis device 100 according to an embodiment of the inventive concept.

Referring to FIG. 1, the disease diagnosis device 100 according to an embodiment of the inventive concept may include a lung cancer diagnosing apparatus. The disease diagnosis device 100 according to an embodiment of the inventive concept may detect volatile organic compounds (hereinafter, referred to as VOCs) to diagnose lung cancer. For example, the disease diagnosis device 100 according to an embodiment of the inventive concept may include a first pre-treatment portion 10, a VOC detection portion 20, and a pump 30.

The first pre-treatment portion 10 may be connected between an inlet 19 of a first intake line 11 and the VOC detection portion 20. The first pre-treatment portion 10 may treat a respiratory gas 110 introduced through the first intake line 11 before detecting the VOCs. The respiratory gas 110 may include moisture, bad breath (e.g., H₂S, H₂, and CH₃SH), non-polar VOCs (e.g., benzene and hexene), and polar VOCs (e.g., toluene, alcohol, and acetone) of a human body (e.g., a person to be tested). However, the embodiment of the inventive concept is not limited thereto. The first pre-treatment portion 10 may remove the moisture and the bad breath (e.g., H₂S, H₂, and CH₃SH) in the respiratory gas 110. For example, the first pre-treatment portion 10 may include a first chamber 12, a first heater 14, a moisture filter 16, and a bad breath filter 18.

The first chamber 12 may surround the first heater 14, the moisture filter 16, and the bad breath filter 18 and protect the same. For example, the first chamber 12 may include metal or plastic. However, the embodiment of the inventive concept is not limited thereto.

The first heater 14 may be disposed in the first chamber 12. The first heater 14 may surround the moisture filter 16 and the bad breath filter 18. The first heater 14 may heat the respiratory gas 110 in the first intake line 11 at a temperature higher than room temperature. When the VOC detection portion 20 does not detect the VOCs, the first heater 14 may heat the moisture filter 16 and the bad breath filter 18 to clean and/or initialize the same. However, the embodiment of the inventive concept is not limited thereto.

The moisture filter 16 may be disposed in the first heater 14 disposed adjacent to the inlet 19 of the first intake line 11. The moisture filter 16 may absorb the moisture in the respiratory gas 110. For example, the moisture filter 16 may include cotton. Alternatively, the moisture filter 16 may include graphite. However, the embodiment of the inventive concept is not limited thereto.

The bad breath filter 18 may be disposed in the first heater 14 between the moisture filter 16 and the VOC detection portion 20. The bad breath filter 18 may remove the bad breath (e.g., H₂S, H₂, and CH₃SH) in the respiratory gas 110. The bad breath filter 18 may include metal powder or metal grains. For example, the bad breath filter 18 may include copper oxide (CuO) or platinum (Pt). Also, the bad breath filter 18 may include metal powder and/or metal grains of selenium oxide (CeO₂) However, the embodiment of the inventive concept is not limited thereto.

A first bypass line 13 may be branched from the inlet 19 of the first intake line 11 and connected to a second intake line 21. The first bypass line 13 may bypass the first pre-treatment portion 10. When a first intake valve 15 of the first intake line 11 is closed, the first bypass line 13 may supply the respiratory gas 110 from the inlet 19 of the first intake line 11 to the VOC detection portion 20 without removing the moisture and the bad breath. For example, the first bypass line 13 may have a first bypass valve 17. The first bypass valve 17 may be opened and closed in an opposite manner to the first intake valve 15. When the moisture and the bad breath in the respiratory gas 110, the first intake valve 15 may be opened, and the first bypass valve 17 may be closed. The respiratory gas 110 may be sequentially supplied into the first pre-treatment portion 10 and the VOC detection portion 20. When the VOC detection portion 20 is cleaned and/or initialized, the first bypass valve 17 may be opened, and the first intake valve 15 may be closed.

The VOC detection portion 20 may be connected between the first pre-treatment portion 10 and the pump 30. The VOC detection portion 20 may detect the non-polar VOCs (e.g., benzene and hexene) and the polar VOCs (e.g., toluene, alcohol, and acetone) in the respiratory gas 110. The VOC detection portion 20 may include a second chamber 22, a second heater 24, and a sensor array 26.

The second chamber 22 may surround the second heater 24 and the sensor array 26. For example, the second chamber 22 may include metal or plastic. However, the embodiment of the inventive concept is not limited thereto.

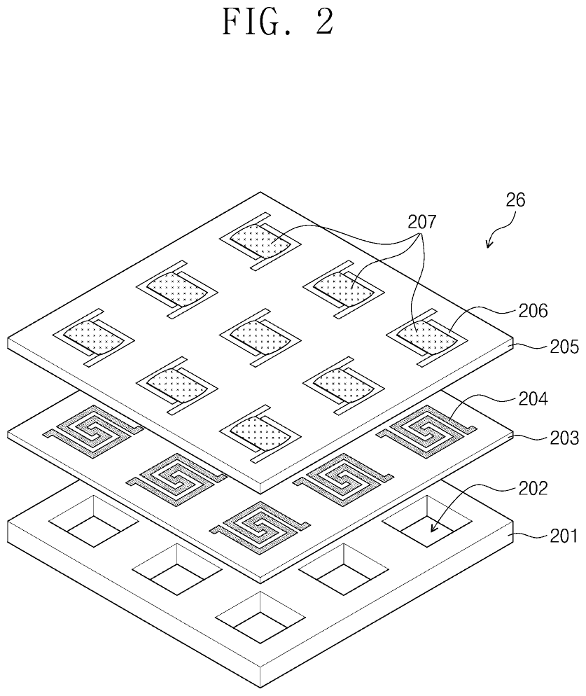

The second heater 24 may be disposed in the second chamber 22. The second heater 24 may surround the sensor array 26. The second heater 24 may heat the sensor array 26 to activate a sensing layer 207 (refer to FIG. 2) of the sensor array 26. Alternatively, the second heater 24 may heat the sensor array 26 to clean and initialize the sensor array 26. However, the embodiment of the inventive concept is not limited thereto.

The sensor array 26 may be disposed in the second heater 24. The sensor array 26 may detect the non-polar VOCs (e.g., benzene and hexene) and the polar VOCs (e.g., toluene, alcohol, and acetone). A control portion (not shown) may determine lung cancer of the human body in the respiratory gas 110 by using a signal for detecting the non-polar VOCs and the polar VOCs of the sensor array 26.

FIG. 2 is a view illustrating an example of the sensor array 26 in FIG. 1.

Referring to FIG. 2, the sensor array 26 may include a substrate 201, a lower insulation layer 203, heater electrodes 204, an upper insulation layer 205, sensing electrodes 206, and sensing layers 207.

The substrate 201 may include a silicon wafer. For example, the substrate 201 may have a plurality of holes 202. The holes 202 may expose a portion of the lower insulation layer 203 on the substrate 201. For example, the plurality of holes 202 may be arranged in the form of a 3×3 matrix. Furthermore, the plurality of holes 202 may have a shape of an N×N matrix. Here, the N may be a natural number.

The lower insulation layer 203 may be disposed on the substrate 201. The lower insulation layer 203 may include silicon oxide or silicon nitride.

The heater electrodes 204 may be disposed on the lower insulation layer 203. The heater electrodes 204 may be disposed on the holes 202. The heater electrodes 204 may heat the sensing electrodes 206 and the sensing layers 207. For example, the heater electrodes 204 may heat the sensing layers 207 at a temperature of about 200° C. to about 250° C. Each of the heater electrodes 204 may include a nickel chromium alloy.

The upper insulation layer 205 may be disposed on the heater electrodes 204 and the lower insulation layer 203. The upper insulation layer 205 may include silicon oxide or silicon nitride. However, the embodiment of the inventive concept is not limited thereto.

The sensing electrodes 206 may be disposed on the upper insulation layer 205. The sensing electrodes 206 may be aligned to the heater electrodes 204. The sensing electrodes 206 may include metal such as gold (Au), silver (Ag), copper (Cu), aluminum (Al), tungsten (W), molybdenum (Mo), indium (In), or cobalt (Co). However, the embodiment of the inventive concept is not limited thereto.

The sensing layer 207 may be connected between one pair of sensing electrodes 206. The sensing layer 207 may detect the non-polar VOCs (e.g., benzene and hexene) and the polar VOCs (e.g., toluene, alcohol, and acetone) in the respiratory gas 110. The sensing layer 207 may include metal oxide such as SnO₂, WO₃, In₂O³, ZnO, Fe₂O₃, Zr₂O₃, and Co₃O₄, graphene, or a carbon nanotube. However, the embodiment of the inventive concept is not limited thereto. The plurality of sensing layers 207 may be the same as or different from each other.

Referring to FIG. 1 again, the pump 30 may be connected to the first pre-treatment portion 10 and the VOC detection portion 20. The pump 30 may be connected to the first intake line 11, the first bypass line 13, and the second intake line 21. The pump 30 may pump the respiratory gas 110 in the first intake line 11, the first bypass line 13, the first pre-treatment portion 10, the second intake line 21, and the VOC detection portion 20. The pump 30 may pump the respiratory gas 110 at a pressure less than the atmospheric pressure.

A second intake valve 35 may be connected between the pump 30 and the VOC detection portion 20. The second intake valve 35 may control the respiratory gas 110 supplied in the pump 30. Although not shown, the second intake valve 35 may be coupled to the second intake line 21 disposed between the VOC detection portion 20 and the first pre-treatment portion 10. However, the embodiment of the inventive concept is not limited thereto.

Resultantly, the disease diagnosis device 100 according to an embodiment of the inventive concept may remove the moisture and the bad breath (e.g., H₂S, H₂, and CH₃SH) in the respiratory gas 110 by using the first pre-treatment portion 10 to increase an efficiency for detecting the VOCs.

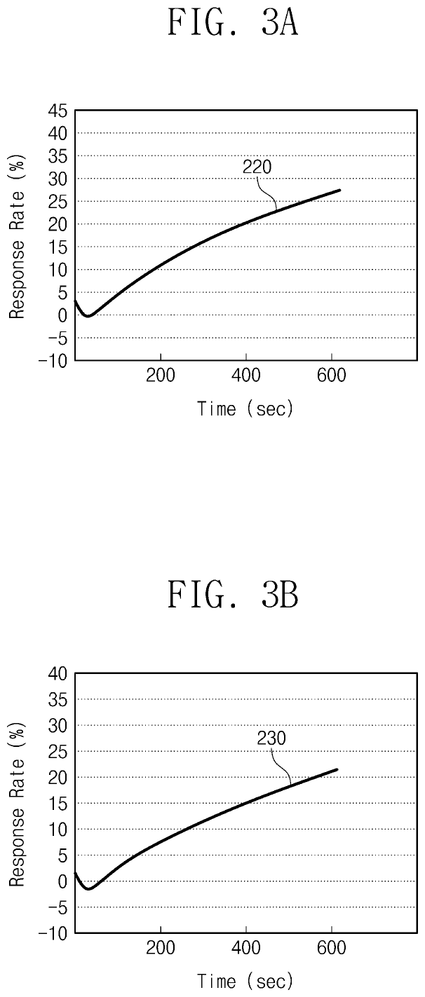

FIGS. 3A and 3B are graphs showing a lung cancer signal 220 or a healthy signal 230 of the respiratory gas 110. In FIGS. 3A and 3B, a horizontal axis represents time, and a vertical axis represents a response rate.

Referring to FIGS. 3A and 3B, the lung cancer signal 220 may have a response rate greater than that of the healthy signal 230. The lung cancer signal 220 may have a response rate of about 27% on the VOCs at a time of about 600 seconds and a relatively high time-varying sensor signal characteristic, and the healthy signal 230 may have a response rate of about 22% on the VOCs at a time of about 600 seconds and a relatively low time-varying sensor signal characteristic. Thus, the control portion may compare a response rate of the detection signal of the respiratory gas 110 with the response rates of the lung cancer signal 220 and the healthy signal 230 to determine the lung cancer of the human body.

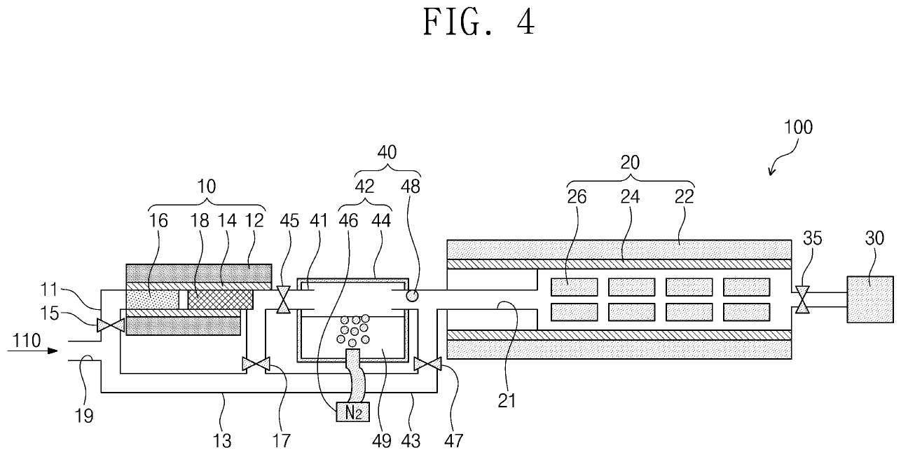

FIG. 4 is a view illustrating an example of the disease diagnosis device 100 according to an embodiment of the inventive concept.

Referring to FIG. 4, the disease diagnosis device 100 according to an embodiment of the inventive concept may further include a second pre-treatment portion 40. The second pre-treatment portion 40 may be connected between the first pre-treatment portion 10 and the VOC detection portion 20. The second pre-treatment portion 40 may control moisture of the respiratory gas 110. For example, the second pre-treatment portion 40 may adjust the moisture of the respiratory gas 110 to reference moisture and increase reliability for detecting the VOCs. The reference moisture may be about 40% to about 60%. When the number of detecting the VOCs of the respiratory gas 110 is increased, a moisture absorption force of the moisture filter 16 may be decreased. When the moisture absorption force of the moisture filter 16 is decreased, the moisture of the respiratory gas 110 may be gradually increased. When the moisture of the respiratory gas 110 is increased, the efficiency for detecting the VOCs may be decreased. Thus, the second pre-treatment portion 40 may constantly maintain the moisture of the respiratory gas 110 regardless of the moisture absorption force of the moisture filter 16 and increase the reliability for detecting the VOCs. For example, the second pre-treatment portion 40 may include a water bubbler 42 and a hygrometer 48.

The water bubbler 42 may be disposed between the first pre-treatment portion 10 and the VOC detection portion 20. The water bubbler 42 may vaporize DI water 49 in the respiratory gas 110 to adjust the moisture in the respiratory gas 110. For example, the water bubbler 42 may include a water bath 44 and a first nitrogen supply portion 46. The water bath 44 may store the DI water 49. The first nitrogen supply portion 46 may supply a nitrogen gas N2 into the DI water 49 of the water bath 44 and generate vapor of the DI water 49. When the vapor is increased, the moisture of the respiratory gas 110 may be increased. When the vapor is decreased, the moisture of the respiratory gas 110 may be decreased.

The hygrometer 48 may measure the moisture of the respiratory gas 110. The control portion may determine the moisture of the respiratory gas 110 by using a moisture measurement signal of the hygrometer 48. The control portion may adjust the moisture of the respiratory gas 110 to the reference moisture and increase the reliability for detecting the VOCs.

A third intake line 41 may be connected between the first pre-treatment portion 10 and the second pre-treatment portion 40. The third intake line 41 may transfer the respiratory gas 110 between the first pre-treatment portion 10 and the second pre-treatment portion 40. The third intake line 41 may have a third intake valve 45. The third intake valve 45 may control and/or adjust the respiratory gas 110 in the third intake line 41.

The second bypass line 43 may be branched from the first bypass line 13 and connected to the second intake line 21. The second bypass line 43 may bypass the second pre-treatment portion 40. The second bypass line 43 may supply the respiratory gas 110 to the VOC detection portion 20 without moisture adjustment. The second bypass line 43 may have a second bypass valve 47. The second bypass valve 47 may be opened and closed in an opposite manner to the third intake valve 45. When the moisture of the respiratory gas 110 is adjusted to the reference moisture, the third intake valve 45 may be opened, and the second bypass valve 47 may be closed. When the moisture of the respiratory gas 110 is not adjusted, the second bypass valve 47 may be opened, and the third intake valve 45 may be closed.

The first intake line 11, the VOC detection portion 20, the pump 30, and the second intake valve 35 may be configured as same as those in FIG. 1.

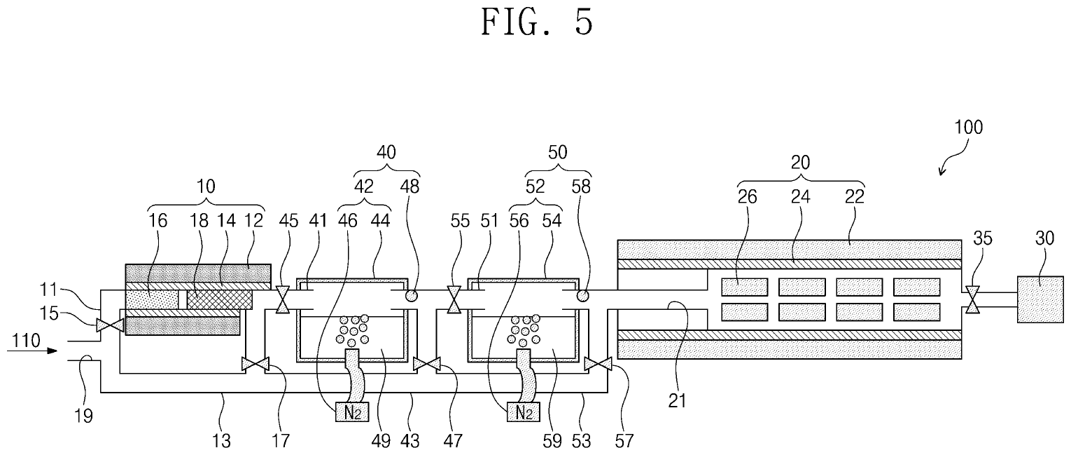

FIG. 5 is a view illustrating an example of the disease diagnosis device 100 according to an embodiment of the inventive concept.

Referring to FIG. 5, the disease diagnosis device 100 according to an embodiment of the inventive concept may further include a third pre-treatment portion 50. The third pre-treatment portion 50 may be connected between the second pre-treatment portion 40 and the VOC detection portion 20. The third pre-treatment portion 50 may adjust a concentration of the non-polar VOCs (e.g., benzene and hexene) in the VOCs of the respiratory gas 110 to a reference concentration and increase the efficiency for detecting the VOCs. For example, the reference concentration may be about 0.1 ppm to about 0.5 ppm. The VOC detection portion 20 may detect the polar VOCs (e.g., toluene, alcohol, and acetone) to determine the lung cancer. The control portion may obtain a concentration of the polar VOCs (e.g., toluene, alcohol, and acetone) by using a detection signal to determine the lung cancer of the human body. The lung cancer may be determined in proportional to the concentration of the polar VOCs (e.g., toluene, alcohol, and acetone). For example, the third pre-treatment portion 50 may include a benzene bubbler 52 and a non-polar VOC gas sensor 58.

The benzene bubbler 52 may be connected between the second pre-treatment portion 40 and the VOC detection portion 20. The benzene bubbler 52 may adjust a concentration of the non-polar VOCs (e.g., benzene and hexene) in the respiratory gas 110 to a reference concentration or remove non-polar materials interfering a reaction by vaporizing a benzene solution 59. When the concentration of the non-polar VOCs is adjusted, the efficiency for detecting the VOCs may be increased. For example, the benzene bubbler 52 may include a benzene bath 54 and a second nitrogen supply portion 56.

The benzene bath 54 may be connected between the third intake line 41 and the second intake line 21. The benzene bath 54 may store the benzene solution 59.

The second nitrogen supply portion 56 may supply a nitrogen gas N2 into the benzene solution 59 of the benzene bath 54 and generate a benzene gas. The benzene gas may adjust the concentration of the non-polar VOCs in the respiratory gas 110.

The non-polar VOC gas sensor 58 may be disposed in the second intake line 21 adjacent to the benzene bath 54. The non-polar VOC gas sensor 58 may detect the non-polar VOCs in the respiratory gas 110. The non-polar VOC gas sensor 58 may include a tungsten oxide (WO₃) sensor. The VOC detection portion 20 may include a polar VOC sensor array. For example, the sensing layer 207 of the sensor array 26 may be a polar VOC sensing layer. The sensing layer 207 may include SnO₂, In₂O₃, ZnO, Fe₂O₃, Zr₂O₃, and Co₃O₄. However, the embodiment of the inventive concept is not limited thereto. The control portion may detect the concentration of the non-polar VOCs in the respiratory gas 110 by using the signal for detecting the non-polar VOCs. The control portion may adjust the concentration of the non-polar VOCs in the respiratory gas 110 to the reference concentration and increase the reliability for detecting the polar VOCs.

A fourth intake line 51 may be connected between the second pre-treatment portion 40 and the third pre-treatment portion 50. The fourth intake line 51 may transfer the respiratory gas 110 between the second pre-treatment portion 40 and the third pre-treatment portion 50. The fourth intake line 51 may have a fourth intake valve 55. The fourth intake valve 55 may be connected to the fourth intake line 51 adjacent to the second bypass line 43. The fourth intake valve 55 may control the respiratory gas 110 in the fourth intake line 51.

The third bypass line 53 may be connected between the second bypass line 43 and the second intake line 21. The third bypass line 53 may be branched from the second bypass line 43 and connected to the second intake line 21 by bypassing the third pre-treatment portion 50. The third bypass line 53 may bypass the third pre-treatment portion 50 and supply the respiratory gas 110 to the VOC detection portion 20. The third bypass line 53 may have a third bypass valve 57. The third bypass valve 57 may be opened and closed in an opposite manner to the fourth intake valve 55. When the concentration of the non-polar VOCs in the respiratory gas 110 is adjusted to the reference concentration, the fourth intake valve 55 may be opened, and the third bypass valve 57 may be closed. The respiratory gas 110 may be supplied to the third pre-treatment portion 50.

When the concentration of the non-polar VOCs in the respiratory gas 110 is not adjusted to the reference concentration, the third bypass valve 57 may be opened, and the fourth intake valve 55 may be closed. The respiratory gas 110 may bypass the third pre-treatment portion 50 and be supplied into the VOC detection portion 20.

The first pre-treatment portion 10, the first intake line 11, and the first bypass line 13 may be configured as same as those in FIGS. 1 and 4.

Hereinafter, a disease diagnosis method using the disease diagnosis device 100 according to an embodiment of the inventive concept will be described.

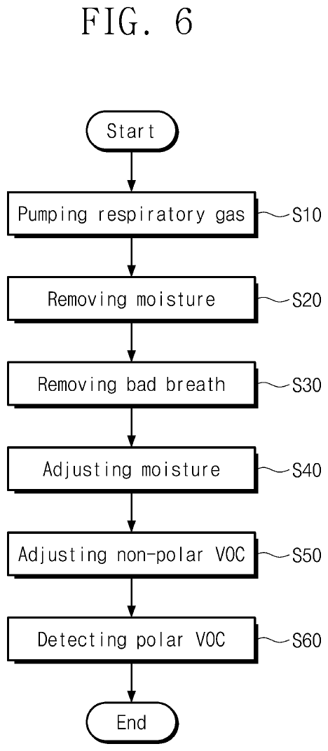

FIG. 6 is a flowchart showing the disease diagnosis method according to an embodiment of the inventive concept.

Referring to FIG. 6, a pump 30 pumps a respiratory gas 110 through a first intake line 11 and a second intake line 21 in a process S10. The pump 30 may pump the respiratory gas 110 at a pressure less than the atmospheric pressure. A first intake valve 15 and a second intake valve 35 may be opened, and a first bypass valve 17 may be closed. The respiratory gas 110 may be sequentially supplied to a moisture filter 16 and a bad breath filter 18 of a first pre-treatment portion 10.

Thereafter, the moisture filter 16 may absorb moisture in the respiratory gas 110 in a process S20. Although the moisture filter 16 may include cotton, the embodiment of the inventive concept is not limited thereto.

Thereafter, the bad breath filter 18 absorbs bad breath in the respiratory gas 110 in a process S30. The bad breath filter 18 may include copper oxide or platinum metal powder and/or metal grains. The respiratory gas 110 may be supplied into a second pre-treatment portion 40 through a third intake line 41. A third intake valve 45 may be opened, and a second bypass valve 47 may be closed.

Thereafter, the second pre-treatment portion 40 may adjust moisture of the respiratory gas 110 to reference moisture in a process S40. The second pre-treatment portion 40 may include a water bubbler 42 and a hygrometer 48. The water bubbler 42 may provide vapor of DI water 49 into the respiratory gas 110. The hygrometer 48 may measure the moisture of the respiratory gas 110. The control portion may adjust the moisture of the respiratory gas 110 to the reference moisture by using a moisture detection signal and increase reliability for detecting VOCs. For example, the reference moisture may be about 45% to about 55%. The respiratory gas 110 may be supplied into a third pre-treatment portion 50 through a fourth intake line 51. A fourth intake valve 55 may be opened, and a third bypass valve 57 may be closed.

Thereafter, the third pre-treatment portion 50 adjusts benzene in the respiratory gas 110 to a reference benzene concentration in a process S50. For example, the third pre-treatment portion 50 may include a benzene bubbler 52 and a non-polar VOC gas sensor 58. The benzene bubbler 52 may supply a benzene gas of a benzene solution 59 into the respiratory gas 110. The non-polar VOC gas sensor 58 may detect non-polar VOCs in the respiratory gas 110. The control portion may detect a concentration of the non-polar VOCs in the respiratory gas 110 to a reference concentration by using a signal for detecting the non-polar VOCs and increase an efficiency for detecting polar VOCs. For example, the reference concentration may be about 0.1 ppm to about 0.5 ppm. The respiratory gas 110 may be supplied to a sensor array 26 of a VOC detection portion 20 through the second intake line 21.

Finally, the sensor array 36 detects the polar VOCs in the respiratory gas 110 in a process S60. The control portion may determine lung cancer of a human body by using a signal for detecting the polar VOCs. The sensor array 26 may include a sensing layer 207 of metal oxide such as SnO₂, In₂O₃, ZnO, Fe₂O₃, Zr₂O₃, and Co₃O₄.

As described above, the disease diagnosis device according to the embodiment of the inventive concept may remove the moisture and the bad breath by using the first pre-treatment portion including the moisture filter and the bad breath filter to increase the reliability for detecting the VOCs in the respiratory gas.

Although the embodiments of the present invention have been described, it is understood that the present invention should not be limited to these embodiments but various changes and modifications can be made by one ordinary skilled in the art within the spirit and scope of the present invention as hereinafter claimed.

What is claimed is:

1. A disease diagnosis device comprising:
a pump configured to pump a respiratory gas;
a first pre-treatment portion connected to the pump and configured to remove moisture and bad breath in the respiratory gas; and
a volatile organic compound (VOC) detection portion connected between the first pre-treatment portion and the pump to detect VOCs in the respiratory gas,
wherein the first pre-treatment portion comprises:
a first chamber;
a moisture filter disposed in the first chamber to remove the moisture; and
a bad breath filter disposed between the moisture filter and the VOC detection portion to remove the bad breath.

2. The disease diagnosis device of claim 1, wherein the bad breath filter comprises metal powder.

3. The disease diagnosis device of claim 2, wherein the metal powder comprises copper oxide or platinum.

4. The disease diagnosis device of claim 1, wherein the moisture filter comprises cotton.

5. The disease diagnosis device of claim 1, wherein the first pre-treatment portion further comprises a first heater disposed in the first chamber and surrounding the moisture filter and the bad breath filter.

6. The disease diagnosis device of claim 1, further comprising a second pre-treatment portion disposed between the first pre-treatment portion and the VOC detection portion to adjust moisture of the respiratory gas,
wherein the second pre-treatment portion comprises:
a water bubbler configured to vaporize DI water; and
a hygrometer disposed between the water bubbler and the VOC detection portion to detect moisture of the respiratory gas.

7. The disease diagnosis device of claim 6, wherein the water bubbler comprises:
a water bath configured to store the DI water; and
a first nitrogen supply portion connected to the water bath and configured to generate vapor of the DI water by supplying a nitrogen gas into the DI water.

8. The disease diagnosis device of claim 6, further comprising a third pre-treatment portion disposed between the second pre-treatment portion of the VOC detection portion to adjust non-polar VOCs of the respiratory gas,
wherein the third pre-treatment portion comprises:
a benzene bubbler configured to vaporize a benzene solution; and
a non-polar VOC gas sensor disposed between the benzene bubbler and the VOC detection portion to detect the non-polar VOCs of the VOCs.

9. The disease diagnosis device of claim 8, wherein the benzene bubbler comprises:
a benzene bath configured to store the benzene solution; and
a second nitrogen supply portion connected to the benzene bath and configured to generate the benzene gas by supplying a nitrogen gas into the benzene solution.

10. The disease diagnosis device of claim 1, wherein the VOC detection portion comprises a polar VOC sensor array.

11. A disease diagnosis device comprising:
a pump configured to pump a respiratory gas;
a first intake line connected to the pump;
a first pre-treatment portion connected between the first intake line and the pump and configured to remove moisture and bad breath in the respiratory gas;
a volatile organic compound (VOC) detection portion connected between the first pre-treatment portion and the pump to detect VOCs in the respiratory gas;
a second intake line configured to connect the first pre-treatment portion to the VOC detection portion; and
a first bypass line branched from the first intake line and connected to the second intake line to bypass the first pre-treatment portion.

12. The disease diagnosis device of claim 11, wherein the first intake line has a first intake valve,
the first bypass line has a first bypass valve that is opened and closed in an opposite manner to the first intake valve, and
the disease diagnosis device further comprises a second intake valve disposed between the pump and the VOC detection portion.

13. The disease diagnosis device of claim 11, further comprising:
a second pre-treatment portion disposed between the first pre-treatment portion and the VOC detection portion;
a third intake line disposed between the first pre-treatment portion and the second pre-treatment portion; and
a second bypass line branched from the first bypass line and connected to the second intake line to bypass the second pre-treatment portion.

14. The disease diagnosis device of claim 13, further comprising:
a third pre-treatment portion disposed between the second pre-treatment portion and the VOC detection portion;
a fourth intake line disposed between the second pre-treatment portion and the third pre-treatment portion; and
a third bypass line branched from the second bypass line and connected to the third intake line to bypass the third pre-treatment portion.

15. The disease diagnosis device of claim 14, wherein the third intake line has a third intake valve,
the second bypass line has a second bypass valve that is opened and closed in an opposite manner to the third intake valve,
the fourth intake line has a fourth intake valve, and
the third bypass line has a third bypass valve that is opened and closed in an opposite manner to the fourth intake valve.

16. A disease diagnosis method comprising:
pumping a respiratory gas;
removing moisture in the respiratory gas by using a moisture filter;
removing bad breath in the respiratory gas by using a bad breath filter; and
determining lung cancer of a human body by detecting volatile organic compounds (VOCs) in the respiratory gas by using a sensor array.

17. The disease diagnosis method of claim 16, further comprising adjusting the moisture of the respiratory gas to reference moisture by vaporizing DI water.

18. The disease diagnosis method of claim 17, wherein the reference moisture is about 40% to about 60%.

19. The disease diagnosis method of claim 16, further comprising adjusting a concentration of non-polar VOCs in the respiratory gas to a reference concentration by vaporizing a benzene solution.

20. The disease diagnosis method of claim 19, wherein the reference concentration is about 0.1 ppm to about 0.5 ppm.
